(19) United States
(12) Patent Application Publication
(10) Pub. No.: US 2007/0286342 A1
(43) Pub. Date: Dec. 13, 2007

(54) Systems and methods for performing radiosurgery using stereotactic techniques

(76) Inventor: Donald B. Fuller, Rancho Santa Fe, CA (US)

Correspondence Address: GORDON & REES LLP, 101 WEST BROADWAY, SUITE 1600, SAN DIEGO, CA, 92101, US

(21) Appl. No.: 11/652,710

(22) Filed: Jan. 12, 2007

Related U.S. Application Data
(60) Provisional application No. 60/811,862, filed on Jun. 7, 2006.

(51) Int. Cl. A61N 5/10

(52) U.S. Cl. 378/65

(57) ABSTRACT

The present invention relates to a method for performing non-homogeneous radiosurgery to provide optimal doses to the site of an abnormal lesion and to minimize damages to surrounding healthy tissues.

### CROSS REFERENCE TO RELATED APPLICATIONS

This application claims priority to provisional application U.S. Ser. No. 60/811,862, which was filed on Jun. 7, 2006.

### TECHNICAL FIELD

The present invention relates to a method for performing non-homogeneous radiosurgery to provide optimal doses to the site of an abnormal lesion and to minimize damages to surrounding healthy tissues.

### BACKGROUND OF THE INVENTION

Radiosurgery is an effective tool for the treatment of abnormal lesions, such as malignant cancers. Stereotactic radiosurgery (“SRS”), combines the principles of stereotaxy (3-D target localization) with multiple cross-fired beams from a high-energy radiation source to precisely irradiate an abnormal lesion within a patient. This technique allows maximally aggressive dosing of the treatment target, while normal surrounding tissue receives lower, non-injurious doses of radiation.

Several SRS systems are available, including cobalt-sourced systems (also known as GAMMA KNIFE®, Elekta Instruments AB, Sweden), a Swedish and linear accelerator (“LINAC”) based devices, such as modified linear accelerators and frameless SRS (e.g., CYBERKNIFE®, Accuray, Sunnyvale, Calif.).

GAMMA KNIFE® employs radioactive cobalt-based gamma ray, whereas LINAC-based systems use X-ray beams generated from a linear accelerator. As a result, the LINAC-based devices do not require or generate any radioactive material. One disadvantage associated with GAMMA KNIFE or conventional LINAC radiosurgery is that a metal head frame is required to be attached to the skull of a patient undergoing brain surgery, and is used to precisely target the radiation beam.

The most advanced LINAC-based system is frameless SRS, which incorporates a miniature linear accelerator mounted on a robotic arm to deliver concentrated beams of radiation to the treatment target from multiple positions and angles. Frameless SRS also employs a real-time x-ray-based image-guidance system to establish the position of the treatment target during treatment, and then dynamically brings the radiation beam into alignment with the observed position of the treatment target. Thus, frameless SRS is able to compensate for patient movement without the need for the invasive and uncomfortable head frame to ensure highly accurate delivery of radiation during treatment. As result, the patient's treatment target receives a cumulative dose of radiation high enough to control or kill the target cells while minimizing radiation exposure to surrounding healthy tissue. With sub-millimeter accuracy, frameless SRS can be used to treat tumors, cancers, vascular abnormalities and functional brain disorders. Frameless SRS can achieve surgical-like outcomes without surgery or incisions. Through the combination of a flexible robotic arm, LINAC, and image guidance technology, frameless SRS is able to reach areas of the body that are unreachable by other conventional radiosurgery systems, including the prostate. When areas of the body outside the brain are targeted by radiosurgery, the technique is sometimes alternatively referred to as SBRT—stereotactic body radiotherapy. The terms “SRS” and “SBRT” have been used interchangeably by different practitioners to describe the same medical procedure.

A second type of radiation treatment is brachytherapy, in which radioactive materials are incorporated into small particles, sometimes referred to as “seeds”, wires and similar related configurations that can be directly implanted in close proximity to the tumor or lesion to be treated. Brachytherapy takes advantage of the simplest physical property of radiation. High doses of radiation are present in the vicinity of the radioactive material, but the dose decreases with the square of the distance from the source. A variety of brachytherapy techniques have bee developed and are in current practice. However, the basic steps of the operation are consistent. Implantation is almost always performed as minor outpatient surgery under general or spinal anesthesia. A prostate brachytherapy procedure typically requires approximately one hour, and patients can return home after a brief recovery period. In an effort to achieve optimal placement of the implanted radiation sources, templates are almost universally used, in contrast to the freehand approach commonly used with other methods of implantation.

Radiosurgery differs from conventional radiotherapy in several ways. The efficacy of radiotherapy depends primarily on the greater sensitivity of tumor cells to radiation in comparison to normal tissue. With all forms of standard radiotherapy, the spatial accuracy with which the treatment is focused on the tumor is less critical compared with radiosurgery; because normal tissues are protected by administering the radiation dose over multiple sessions (fractions) that take place daily for a period of a few weeks. This form of radiation is more effectively repaired by normal tissues, whereas radiosurgery is far more likely to ablate all normal tissues in the high dose zone. As such, radiosurgery, by its very definition, requires much greater targeting accuracy. With Stereotactic Radiosurgery (SRS), normal tissues are protected by both selective targeting of only the abnormal lesion, and by using cross-firing techniques to minimize the exposure of the adjacent anatomy. Since highly destructive doses of radiation are used, any normal structures (such as nerves or sensitive areas of the brain) within the targeted volume are subject to damage as well. Typically, SRS is administered in one to five daily fractions over consecutive days. Nearly all SRS is given on an outpatient basis without the need for anesthesia. Treatment is usually well tolerated, and only very rarely interferes with a patient's quality of life. Accordingly, SRS has been used to treat benign and malignant tumors, vascular malformations, and other disorders with minimal invasiveness.

Radiosurgical treatment generally involve several phases. First, a three-dimensional map of the anatomical structures in the treatment area is constructed using an imaging technique, such as positron emission tomography (PET) scanning, single photon emission computed tomography (SPECT), perfusion imaging, tumor hypoxia mapping, angiogenesis mapping, blood flow mapping, cell death mapping, computed tomography (CT), or magnetic resonance imaging (MRI). Next, a treatment plan is developed to deliver a dose distribution according to the three-dimensional map. Finally, a patient is treated according to the treatment plan with an appropriate radiosurgical technique.

In accordance with presently used technology, irradiating a particular target area of a patient, such as a tumor, is planned with computer assistance, and then performed on the basis of the planning, using computer-guided irradiation devices. Generally, imaging methods, such as computer tomography or nuclear spin tomography, are used to determine the outer contours of the region to be irradiated, such as an outer contour in most cases being marked on the tomographic images obtained. An irradiation target determined in this way is generally irradiated as homogeneously as possible in accordance with conventional irradiation technology, wherein it is, in principle, unimportant whether the planning performed beforehand is performed inversely or conventionally.

In conventional planning, a particular irradiation target is simply selected and the dosage with which the area is to be irradiated is established. Irradiation is then performed accordingly. In inverse irradiation planning, the dosage is determined or prescribed differently. Generally, histograms (dosage-volume histograms) are used. Since in most cases perfect homogeneity cannot technically be achieved without the risk of damaging normal structures, the dosage can be prescribed, for example, in accordance with the following approach: 80% of the volume of the tumor can be irradiated with at least 90% of the prescribed dosage, 95% of the volume of the tumor can be irradiated with at least 60% of the prescribed dosage, etc.

One problem with such conventional irradiation planning is that the irradiation target area is treated as homogeneous. In the case of prostate cancers, for example, the whole prostate is treated in a homogeneous fashion without distinguishing the tumor cells from the surrounding healthy cells. In addition, tumors often exhibit regions of higher activity and/or aggressiveness as well as regions of low activity and/or aggressiveness.

Accordingly, there is a need to develop a non-homogeneous treatment plan according to the non-homogeneity of the treatment target to provide optimal doses to the target and to minimize damages to the healthy surrounding cells, raising the dose to the typically most heavily involved area, with simultaneous limitation of the dose in target volume regions less likely to harbor a heavy malignant cell burden. In the case of prostate cancer, more involved and less involved regions within the prostate may be reasonably described as the peripheral zone and the periurethral area, respectively.

### SUMMARY OF THE INVENTION

The present invention relates to a method for performing non-homogeneous radiosurgery to provide optimal doses to the site of an abnormal lesion and to minimize damages to non-targeted healthy tissues. The method comprises the steps of taking an image of a targeted area of a patient, developing a treatment plan based on the image of the targeted area with dose non-homogeneity to provide sufficient doses to the targeted lesion and to minimize damage to the surrounded and surrounding healthy tissues around the targeted area, and treating the patient with a radiosurgical technique.

### BRIEF DESCRIPTION OF THE DRAWINGS

FIG. 1 shows a comparison of CYBERKNIFE® Virtual HDR (Non-homogeneous Frameless SRS, 1A) vs. real HDR brachytherapy (1B) for prostate cancer.

FIG. 2 shows a comparison of CYBERKNIFE® Virtual HDR (left) versus actual HDR brachytherapy (right) mid-axial dose plot. The planning target volume (PTV), covering the prostate and a small margin of adjacent tissue, is indicated by light shading, within the heavy solid line. Each black star in the right hand figure represents a HDR catheter—A tube that protrudes through the perineum to act as a conduit to deliver a radioactive source to the PTV within the prostate. The urethra is the central darkly shaded oval structure. The dashed line represents 100% of prescribed radiation dose; dotted line represents 125%, and thin internal solid lines represent 150%. The heaviest concentration of cancer cells lies within the peripheral zone of the prostate, which is approximately bounded by the 125% isodose line in each figure.

### DETAILED DESCRIPTION OF THE INVENTION

As used in this disclosure, the singular forms “a”, “an”, and “the” may refer to plural articles unless specifically stated otherwise. Furthermore, the use of grammatical equivalents of articles is not meant to imply differences among these terms unless specifically indicated in the context. Unless defined otherwise, all technical and scientific terms used herein generally have the same meaning as commonly understood by one of ordinary skill in the art to which this invention belongs.

Brachytherapy refers to a procedure in which radioactive seeds or sources of radioactivity are placed in or near the tumor, giving a high radiation dose to the tumor while reducing the radiation exposure in the surrounding healthy tissues. It is also known as implant radiation, internal radiation, or interstitial radiation. High Dose Rate (HDR) brachytherapy describes the use of a single high-intensity radioactive source that “steps” through hollow tubes that traverse the tumor volume, stopping at optimized “dwell positions” within the tubes, and residing at these dwell positions for computer programmable variable amounts of time, to optimize radiation dose distribution.

Computed tomography (CT) refers to an imaging method in which the region of interest is imaged by taking serial, parallel sections of the region at regular intervals followed by digital reassembly of the sections to provide a three dimensional image. CT can be used to image soft tissue, bone, vasculature and implanted seeds. CT scanners are available from a number of vendors known to those skilled in the art, including General Electric, Philips and Siemens.

D value refers to a dosage of radioactivity received by a specific percentage of the target volume. For example, D90 means that 90% of the target volume has received a given dose. This number is obtained by a computer calculation of the radiation isodose line that covers exactly 90% of the contoured target volume.

Gray (Gy) refers to a dose of radiation equal to 100 rads, or centigray (cGy).

Planning target volume (“PTV”) refers to the volume of a treatment target plus appropriate margin as determined using 3-dimensional imaging methods. There is some variance in the volume determined using various imaging methods.

The present invention provides a method for performing non-homogeneous radiosurgery to deliver optimal doses to the site of an abnormal lesion, thus minimizing damages to surrounding healthy tissues. The method comprises the steps of constructing three-dimensional anatomical images of the treatment target (target volume) of a patient using one or more imaging techniques, developing a treatment plan with a predefined non-homogeneity according to the anatomical images for delivering a predefined dose distribution to the target volume, and treating the patient with a radiosurgical technique according to the treatment plan.

Various imaging techniques are suitable for use in the present invention for generating three-dimensional anatomical or physiological images, including CT, PET scanning, SPECT, diffusion imaging, perfusion imaging, tumor hypoxia mapping, angiogenesis mapping, blood flow mapping, cell death mapping, magnetic resonance imaging (MRI), X-rays, and ultrasonic imaging methods. Two or more images from different sources or different times during the treatment may be viewed together using a fusion software program to improve registration and fusion of anatomic and physiological images. Such registration may use fixed points in the acquired image or radiopaque locators (or fiducials) that are implanted in the patient for this purpose. It should be understood by skilled persons in the art that there are many ways to capture images of lesions within a human body. Therefore, this invention should not be limited to any particular type of imaging system. One important aspect of the invention is that the imaging system is capable of identifying the contours of the target volume, along with normal tissues (i.e., surrounded tissue and surrounding tissue) and critical structures.

Typically, an imaging method is chosen that will contrast a particular lesion with surrounding tissue or that measures a parameter that characterizes the lesion. As an example, in fluorodexoyglucose (FDG) PET imaging, radiolabelled glucose is preferentially taken up by tumor cells. As another example, PROSTASCINT® binds to prostate specific membrane antigen (“PSMA”), which is overexpressed in prostate cancer. Other non-image clinical data regarding the patient may also be imported for use in later analysis. As an example, for the prostate cancer surgical planning application, ProstaScint-based isodose contour data, the serum PSA value, the biopsy Gleason score value, and the clinical stage can be combined in a prognostic factor model used to predict the likelihood of extra-capsular extension or cavernous nerve involvement. As another example, for using changes in isodose contours and contour relationships to predict the likelihood of lung cancer complete response to chemotherapy, factors such as tumor histology and tumor grade can be integrated into the predictive models.

In generating a treatment plan, the non-homogeneity may be determined based on the shape and/or aggressiveness of the abnormal lesion. The non-homogeneity may be set at no less than 5%, no less than 6%, no less than 8%, no less than 10%, no less than 15%, no less than 20%, no less than 25%, no less than 30%, no less than 40%, or no less than 50%.

The treatment plan may be generated using standard manual planning. Standard manual planning involves a trial-and error approach performed by an experienced physician. For instance, a physician may choose how many isocenters to use, as well as the location in three dimensions, the collimators' size for stereotactic radiosurgery, and the weighting to be used for each isocenter. A treatment planning software program may also be employed to calculate the dose distribution resulting from this preliminary plan. Prospective plans are evaluated by viewing isodose contours superimposed on anatomical images and/or with used quantitative tools such as cumulative dose-volume histograms (“DVH”).

The treatment plan may also be generated using inverse planning, which employs software to optimize the dose distributions specified by physicians based on a set of preselected variables, such as required doses, anatomical data on the patient's body and the target volume, and a set of preselected or fixed beam orientation parameters and beam characteristics for stereotactic radiosurgery. Other parameters, such as (1) number of beams, (2) configuration of beams, (3) beam intensity, (4) initial gantry angle, (5) end gantry angle, (6) initial couch angle, (7) end couch angles, (8) prescription dose, (9) target volume, and (10) set of target points, may also be considered in treatment planning. Additionally, the dosage can be prescribed as 80% of the volume of the treatment target can be irradiated with at least 90% of the prescribed dosage, 95% of the volume of the treatment target can be irradiated with at least 60% of the prescribed dosage. A common radiotherapy or radiosurgery dosimetry benchmark is that >=95% of the planning target volume (PTV) receives >=100% of the prescribed radiation dose.

The treatment plan can be executed using a variety of radiosurgical techniques. In one embodiment, the radiosurgical technique is non-invasive stereotactic radiosurgery, such as, for example, a linear accelerator, a GAMMA KNIFE®, or any other external beam delivery device capable of providing a radiation source. An external beam delivery device may comprise a plurality of external beams having variable intensity, a plurality of collimators for adjusting the size of the beams, and a mechanism for moving the unit with respect to a patient positioned within a stereotactic frame in order to adjust the angle and entry point of each radiation beam.

Various linear accelerator technologies can be used in the present invention, including, but not limited to, three-dimensional conformal radiation therapy (“3DCRT”), non-coplanar arc stereotactic radiosurgery (“NASR”), intensity modulated radiation therapy, intensity modulated arc therapy (“IMAT”), and frameless SRC (e.g., CYBERKNIFE®).

3DCRT involves the use of three-dimensional computer planning systems to geometrically shape the radiation field to ensure adequate coverage of the target volume, while sparing normal tissue. The tools for 3DCRT include patient-specific CT data, beam's-eye-view (“BEV”) treatment planning, and multileaf collimators (“MLC”). Guided by the target contours identified in the CT images, beam orientations are chosen and beam apertures are accurately delineated using BEV. The beam aperture can be fabricated with conventional blocks or defined by MLC. The dose distribution within the field is determined by choice of beam intensity and simple modulators such as wedges and tissue compensators.

Radiosurgery is distinguished from conventional external beam radiation therapy of the central nervous system by its localization and treatment strategy. In radiosurgery, the number of fractions (treatment sessions) is much less, and the dose per fraction is much larger than in conventional radiotherapy. Radiosurgery involves the use of external beams of radiation guided to a desired point within the brain using a precisely calibrated stereotactic frame mechanically fixed to the head, a beam delivery unit, such as a LINAC, GAMMA KNIFE®, and three-dimensional medical imaging technology. For LINAC radiosurgery, the table on which the patient lies and the beam delivery unit is capable of rotating about distinct axes in order to adjust the angle and entry point of a radiation beam. The tissue affected by each beam is determined by the patient's position within the stereotactic frame, by the relative position of the frame in relation to the beam delivery unit, by collimators that adjust the size of the beam, and by the patient's anatomy. Additionally, the intensity of each beam can be adjusted to govern its dose contribution to each point.

In IMRT, the beam intensity is varied across the treatment field. Rather than being treated with a single, large, uniform beam, the patient is treated instead with many very small beams, each of which can have a different intensity. When the tumor is not well separated from the surrounding organs at risk, such as what occurs when a tumor wraps itself around an organ, there may be no combination of uniform intensity beams that can safely separate the tumor from the healthy organ. In such instances, adding intensity modulation allows more accurate conforming of the three-dimensional high dose radiotherapy treatment of the tumor, while limiting the radiation dose to adjacent healthy tissue.

Intensity modulated arc therapy (IMAT) is a form of IMRT that involves gantry rotation and dynamic multileaf collimation. Non-coplanar or coplanar arc paths are chosen to treat the target volume delineated from CT images. The arcs are chosen such that intersecting a critical structure is avoided. The fluence profiles at every 5 degrees are similar to a static IMRT field. As the gantry rotates, the dynamic MLC modulates the intensity to deliver the dose to the target volume while sparing normal tissue. The large number of rotating beams may allow for a more conformal dose distribution than the approach of multiple intensity modulated beams.

In an exemplary embodiment, frameless SRS is used as a radiosurgical method for the treatment of prostate cancer. A patient diagnosed with prostate cancer is selected for prostate radiosurgery using non-homogeneous frameless SRS. The non-homogeneous frameless SRS of the present invention deviates significantly from the current frameless SRS practice, in which the whole prostate with suspected cancer is radiated as homogeneously as possible and the dose variability is typically no greater than 5% with IMRT and no greater than 30% with most conventional radiosurgery protocols. In summary, conventional treatment modalities favor homogeneous radiation dosage.

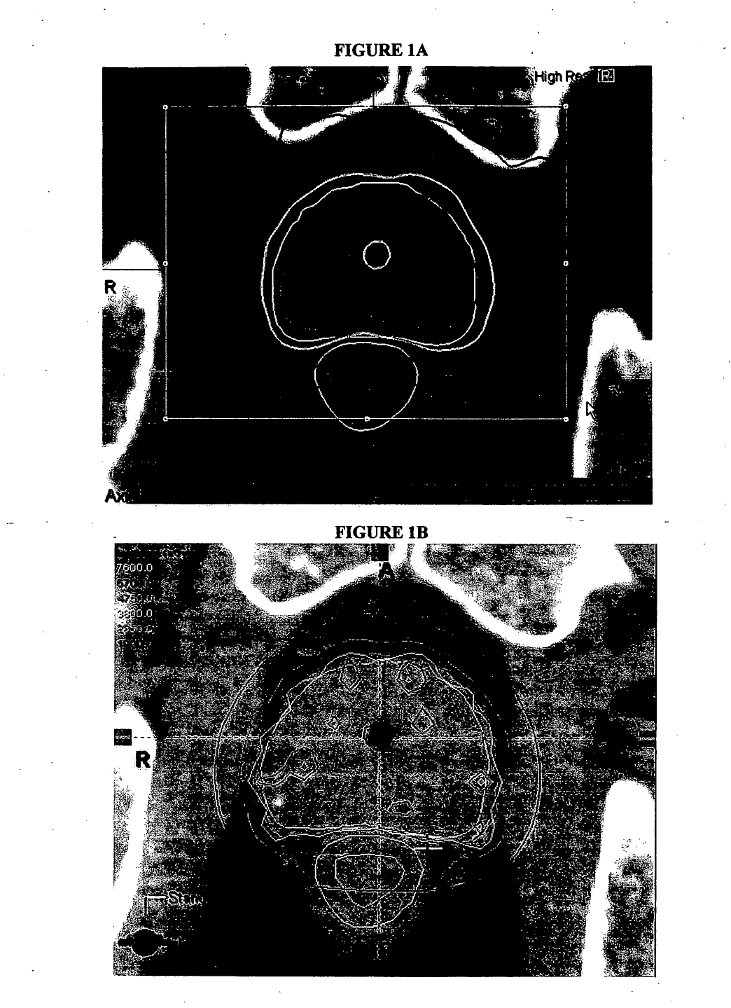

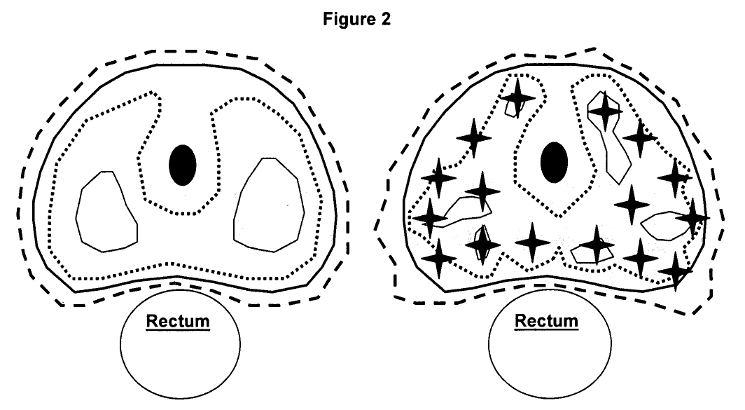

In contrast, the non-homogeneous frameless SRS of the present invention provides a deliberately non-homogeneous dosage of radiation, such as by deliberately introducing dose variability greater than 30% within the planning target volume (PTV). Referring to FIG. 1, the PTV is the area inside the 57% isodose line, which is the prescription dose value of the maximum intraprostatic dose. In FIG. 2, this area is indicated by light shading, within the heavy solid line. This corresponds to a “non-homogeneity” of 100/57, or 175%. For the purpose of the present invention, any value greater than 150% is considered to be “non-homogeneous.”

The non-homogeneity of the radiation method of the present invention can also be expressed as a mean percentage of volume receiving 150% or greater than the prescription dose (“V150”). Referring to FIG. 1, the prescription dose value is 57% of the maximum intraprostatic dose. Accordingly, the 150% isodosage line is represented in red, and is the 86% dose (i.e., 150% of 57%.) The 150% isodosage line is represented by the tin internal solid line in FIG. 2. For the nonhomogeneous frameless SRS, the volume receiving greater than 150%, i.e., the V150, is greater than 1%, and in some cases is greater than 5%, 10%, 15%, 20%, 25%, 30%, 40%, or even 50%.

In the example depicted in FIG. 1, the PTV is optimized to match the highest dose with the greatest cancer cell burden, so that the diseased tissues receive significantly more radiation than the surrounded and surrounding healthy tissues, which more closely resembles the dose distribution typically created by HDR brachytherapy, yet is delivered noninvasively by radiosurgery (also known as “SBRT”, or Stereotactic Body Radiotherapy).

In a more conventional existing treatment protocol for prostate cancer, the specified dose prescription is to the 70% to 90% isodose line, relative to a maximum value of 100%. In this protocol, there is a 20% dose variability. This corresponds to a maximum “non-homogeneity” allowed by this protocol of 100/70, or 143%, and any amount of non-homogeneity above 143% is a “deviation” from this protocol. Thus, any non-homogeneity of 150% or more of the presecription dose is contraindicated by this nationally accepted protocol.

In another embodiment, the treatment technique is brachytherapy. Brachytherapy is especially effective in the treatment of cancer in any tissue which is sufficiently solid to allow for placement of seeds through the skin without making an incision. Variations of brachytherapy can be used in the present invention, including permanent implantation and HDR brachytherapy. In an exemplary embodiment, brachytherapy is used for the treatment of prostate cancer as described in U.S. Published Application 2004/0225174. In general, a patient diagnosed with prostate cancer is selected for prostate brachytherapy. Prior to the operation, a treatment plan is developed based on a series of tests including a stepping ultrasound volume study to determine the contour of the prostate. The non-homogeneity, as expressed as a mean percentage of volume receiving 150% of the prescription dose, V150, is no less than 20%, 40%, 42%, 45%, 50%, 55%, 60%, or 75%.

### EXAMPLE

### Peripheral Zone Dose Escalated CyberKnife Prostate Radiosurgery: Dosimetry Comparison with HDR

Based on successful treatment of prostate cancer with High Dose Rate (HDR) brachytherapy monotherapy, a CyberKnife (CK) IRB-approved protocol (“protocol CK”) was followed, using reported HDR fractionation (38Gy/4fx), deliberately escalating peripheral zone dose. In this study the protocol CK was studied versus the present invention, simulated HDR, for identical prostate volumes in a series of patients, comparing dosimetric endpoints and clinical observations.

Nine consecutive patients treated with CK from July-November 2006 are studied; 8 receiving protocol CK monotherapy and 1 receiving a CK boost. All were normalized to the CK monotherapy protocol dose (38Gy/4fx). Minimum CK PTV V100 requirement was 95%. CK dose constraints: PTV—200% (76Gy); rectal wall—100% (38Gy); rectal mucosa (3 mm rectal wall contraction)—75% (28.5Gy); urethra—120% (45.6Gy); bladder—120% (45.6 Gy); For all patients, a corresponding simulated HDR plan using dicom-transferred common contour sets was designed on the Varian Varisource® HDR computer, using 15-20 simulated HDR catheters, matching median PTV V100 values within 0.8%, minimizing HDR urethra and rectal wall doses as much as possible.

The median CK prescription isodose line was 59% (54-67%). Respective median CK vs. HDR PTV: D90-39.7Gy vs. 41.2Gy; V100-96.4% vs. 95.6%; V125-41.2% vs. 67.7%; V150 6.5% vs. 37.5%. Respective median CK vs. HDR urethra: Dmax—44.3Gy vs. 50.2Gy; D10—41Gy vs. 47.2Gy; D50—38.8Gy vs. 45Gy. Respective median CK vs. HDR bladder: Dmax—42.8Gy vs. 54Gy: D10—29.3Gy vs. 24.1Gy. Respective median CK vs. HDR rectal wall: V80—1.3 cc (0.3-4.0) vs 2.5 cc (0.7-3.3); Dmax—37.3Gy vs. 36.9Gy; D1—33.5Gy vs. 34.9Gy; D10—22.9Gy vs. 25.7Gy; D25—14.8Gy vs. 19.2Gy.

Within the PTV, V100 and D90 appear comparable for CK and HDR, while V125 and V150 are higher for HDR, indicating similar prescription isodose coverage with each modality, and moderately higher estimated uniform dose (EUD) for HDR. Urethra dosimetry values were uniformly lower in CK cases. Matching simulated HDR with actual CK urethra dosimetry invariably caused HDR PTV V100 deviation below the protocol requirement, suggesting that CK better limited urethra dose while maintaining PTV prescription isodose coverage. Bladder dosimetry differences of unknown clinical significance were observed (lower Dmax with CK; lower D10 with HDR). Both modalities created similar rectal wall Dmax, D1 and V80 values, while CK created more rapid dose fall-off with increasing distance beyond the PTV, manifested by increasing disparity trends in rectal wall D10 and D25 parameters, favoring CK. Additional simulated CK iterations in a limited number of patients, allowing higher CK urethra doses (matching comparison HDR values), result in significantly increased CK PTV V125 and V150 values, which more closely approach comparison HDR values, without significantly altering CK rectal wall or bladder dosimetry. This indicates intraprostatic dose sculpting flexibility with CK, highly programmable by the user. Preliminary clinical results are encouraging, with a median 50% one month PSA decrease in the first 5 evaluable CK monotherapy protocol patients, and self limited, primarily urologic side effects.

The examples set forth above are provided to give those of ordinary skill in the art with a complete disclosure and description of how to use the preferred embodiments of the present invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method for performing a non-homogeneous radiosurgery, comprising the steps of
taking images of a treatment target in a patient with one or more imaging methods;
developing a non-homogeneous treatment plan according to the images to provide optimal dosage to the treatment target and minimal dosage to the areas surrounding the treatment target; and
treating the patient with the radiosurgery according to the treatment plan.

2. The method of claim 1, wherein the radiosurgery is a non-invasive stereotactic radiosurgical technique.

3. The method of claim 1, wherein the radiosurgery is frameless stereotactic radiosurgery.

4. The method of claim 1, wherein the radiosurgery is an invasive radiosurgical technique.

5. The method of claim 1, wherein the radiosurgery is a brachytherapeutic technique.

6. The method of claim 1, wherein the non-homogeneity is at least 30%.
